SEQUENCE LISTING (15 sequences)

SEQ ID NO: 1 - protein, 20 aa, Artificial Sequence
  SAP20
SAPDTRPAPGSTAPPAHGVT

SEQ ID NO: 2 - protein, 20 aa, Artificial Sequence
  SAP20
SAPESRPAPGSTAPAAHGVT

SEQ ID NO: 3 - protein, 20 aa, Artificial Sequence
  SAP20
SAPESRPAPGSTAPPAHGVT

SEQ ID NO: 4 - protein, 20 aa, Artificial Sequence
  SAP20
SAPDTRPAPGSTAPAAHGVT

SEQ ID NO: 5 - protein, 21 aa, Artificial Sequence
  H1 to H3
AHGVTSAPDTRPAPGSTAPPA

SEQ ID NO: 6 - protein, 21 aa, Artificial Sequence
  H4 to H6
AHGVTSAPESRPAPGSTAPAA

SEQ ID NO: 7 - protein, 16 aa, Artificial Sequence
  P1
SAPDTRPAPGSTAPPA

SEQ ID NO: 8 - protein, 16 aa, Artificial Sequence
  P2
SAPESRPAPGSTAPAA

SEQ ID NO: 9 - protein, 25 aa, Artificial Sequence
  TAP25
TAPPAHGVTSAPDTRPAPGSTAPPA

SEQ ID NO: 10 - protein, 20 aa, Artificial Sequence
  GST20-AES
GSTAPAAHGVTSAPESRPAP

SEQ ID NO: 11 - protein, 17 aa, Artificial Sequence
  SAP17
SAPDTRPAPGSTAPPAH

SEQ ID NO: 12 - protein, 20 aa, Artificial Sequence
  GVT20
GVTSAPDTRPAPGSTAPPAH

SEQ ID NO: 13 - protein, 23 aa, Artificial Sequence
  GVT23
GVTSAPDTRPAPGSTAPPAHGVT

SEQ ID NO: 14 - protein, 27 aa, Artificial Sequence
  STA27
STAPPAHGVTSAPDTRPAPGSTAPPAH

SEQ ID NO: 15 - protein, 20 aa, Artificial Sequence
  Unit of 100mer
HGVTSAPDTRPAPGSTAPPA

(19) United States
(12) Patent Application Publication
(10) Pub. No.: US 2006/0142546 A1
(43) Pub. Date: Jun. 29, 2006

(54) Immunogenic muc1 glycopeptides

(76) Inventor: Franz-Georg Hanisch, Koln (DE)

Correspondence Address: CLARK & ELBING LLP, 101 FEDERAL STREET, BOSTON, MA, 02110, US

(21) Appl. No.: 10/525,672

(22) PCT Filed: Sep. 5, 2003
(86) PCT No.: PCT/EP03/09882

§ 371 Date: Aug. 10, 2005

(30) Foreign Application Priority Data
Sep. 5, 2002 (DE) 10241207.3
Feb. 11, 2003 (DE) 10305607.6

(51) Int. Cl. C07H 21/04; C12P 21/06; C07K 9/00

(52) U.S. Cl. 530/322; 536/23.5; 435/69.1; 435/320.1; 435/325

(57) ABSTRACT

Provided are novel MUC1 peptides for use in anti-tumor vaccination and methods of producing those peptides. Furthermore, methods of producing a population of autologous antigen presenting cells (APCs) and of producing genetically engineered APCs, which are capable of inducing effective immune responses against MUC1 are described. The described peptides are particularly useful for the treatment of breast cancer or other MUC1-positive carcinomas including colorectal, pancreatic and gastric carcinomas.

### FIELD OF THE INVENTION

The present invention relates to MUC1 peptides and to methods of producing those peptides. The invention further relates to an ex vivo-method of producing a population of autologous antigen presenting cells (APCs) and of producing genetically engineered APCs, which are capable of inducing effective immune responses against MUC1. The invention also relates to APCs, which are obtainable by these methods as well as to the use of the above mentioned peptides and APCs in a pharmaceutical composition for the treatment of breast cancer or other MUC1-positive carcinomas including colorectal, pancreatic and gastric carcinomas.

### BACKGROUND OF THE INVENTION

MUC1 is overexpressed in breast cancer and by many other carcinomas and the tumor-associated glycoform of the mucin is known to expose multiple peptide epitopes within its repeat domain. These immunogenic peptide epitopes make MUC1 a promising tumor antigen with diagnostic as well as therapeutic potential in the treatment of cancer.

The development of effective vaccine and immunotherapies for human cancers and infectious agents often is dependent on the generation of protective immune responses to specific domains of membrane proteins. The tandem repeat (TR) domain of the breast, pancreatic, and ovarian tumor antigen, human mucin MUC1 (Barnd et al., PNAS USA 86 (1989), 7159-7163; Jerome et al., Cancer Res. 51 (1991), 2908-2916), the principal neutralizing domain of HIV-1 (Javaherian et al., PNAS USA 86 (1989), 6768-6772; Javaherian et al., Science 250 (1990), 1590-1593) and the proline rich neutralization domain of the feline leukemia virus external surface unit protein (gp-70) (Nunberg et al., PNAS 81 (1984), 3675-3679; Elder et al., J. Virol. 61 (1987), 8-15; Strouss et al., J. Virol. 61 (1987), 3410-3415; Nick et al., J. Gen. Virol. 71 (1990), 77-83) are examples hereof.

Regarding MUC1, humoral and cellular responses have been demonstrated in cancer patients (Kotera et al., Cancer Res. 54 (1994), 2856-2860; Barnd et al., Proc. Natl. Acad. Sci. USA 86 (1989), 7159-7.163), but also in pregnant woman (Hilkens et al., Cancer Res. 46 (1986), 2582-2587) and healthy individuals (Agrawal et al., Cancer Res. 55 (1995), 2257-2261). Although these natural responses are usually insufficient to fight the progress of cancer, MUC1-derived peptides or glycopeptides are used currently in clinical trials to trigger therapeutically and prophylactically immune reactions in humans (Karanikas et al., J. Clin. Invest. 100 (1997), 2783-2792; Goydos et al., J. Surg. Res. 63 (1996), 298-304).

There is growing evidence that triggering of efficient humoral and CTL responses to MUC1 needs the activation of specific T helper cell clones, which is induced by MHC class II-presented antigen fragments. The generation of MHC class II-restricted peptide epitopes by antigen presenting cells (APCs) like dendritic cells (DCs) follows a multistep process starting with endocytosis, followed by the processing in late endosomal compartments and resulting in the binding of proteolytic peptide fragments to MHC class II proteins and their transport to the cell surface. While many aspects of this complex process have been elucidated there is currently little evidence on the processing and MHC class II presentation of glycosylated antigens, in particular of the highly O-glycosylated mucin antigens. To enable the design of efficient tumor vaccines on the basis of MUC1 knowledge on how DCs or other APCs deal with O-glycosylated peptides is of importance. One particular question in this context refers to the fate of complex O-linked glycans during processing, since efficient peptide fragmentation may afford complete or partial removal of sugars prior to proteolysis. O-linked glycans could also direct the processing with respect to the accessibility of cleavage sites and hence restrict the pattern of peptide fragments on the one hand, while they enrich the pattern of epitopes on the other.

Although several of the cathepsins have been identified as components of the processing machinery (Honey et al., J. Biol. Chem. 276 (2001), 22573-22578; Shen et al., J. Immunol. 158 (1997), 2723-2730), it is currently not known which enzyme(s) are involved in the processing of MUC1 and at which sites within the repeat domain they actually cleave the protein. Expectedly, there are multiple cleavage sites and only subfractions of the generated peptide fragments may fulfil the requirements for binding to MHC class II molecules. Thus, while there is a constant need of specific and immunogenic MUC1 peptides for use as anti-cancer vaccines, so far the structural requirements for designing immunogenic MUC1 peptides had not been elucidated.

The solution to said technical problem is achieved by providing the embodiments characterized in the claims, and described further below.

### SUMMARY OF THE INVENTION

The present invention is directed to novel immunogenic MUC1 peptides, which can be used for immunization in mammals, especially in humans. In particular, peptides of least 9 amino acids in length derived from the tandem repeat domain of MUC1 and having the amino acid sequence SAP at its N-terminus are provided.

The present invention also concerns nucleic acids encoding such peptides and vectors comprising said nucleic acids as well as host cells transfected with nucleic acids or vectors of the invention.

Furthermore, the present invention relates to a method of producing an immunogenic MUC1 peptide, which allows the originally contained glycosylation pattern to be conserved during the production process.

The use of the MUC1 peptides in accordance with the present invention may be accompanied by the use of further therapeutic agents such as toxins and anti-cancer drugs commonly used in the therapy or diagnosis of cancer.

It is another object of the present invention to provide a fusion molecule comprising the peptide of the invention and a functional moiety such as a toxin, label, etc.

It is another object of the present invention to provide a method of producing a population of autologous antigen presenting cells (APCs), which are capable of inducing effective immune responses against MUC1, comprising the steps of
(a) providing autologous APCs from a tumor patient;
(b) contacting the autologous APCs from the tumor patient with an effective amount of a peptide or fusion molecule of the invention under conditions which allow endocytosis, processing and MHC class II presentation of the peptide fragments by said APCs; and
(c) isolating said peptide presenting APCs for the purpose of immunotherapeutic application in the patient.

It is another object of the present invention to provide a method of producing genetically engineered APCs, which are capable of inducing effective immune responses against MUC1, comprising the steps of
(a) providing a nucleic acid encoding at least one of the peptides of the invention or a fusion molecule comprising said at least one peptide;
(b) transfecting the APCs with said nucleic acid; and
(c) selecting APCs, which present said peptides in an MHC II restricted manner.
   APCs obtainable by said method are subject of the present invention as well.

The peptides, fusion molecules, nucleic acids, vectors, APCs, and compositions containing any one of those compounds can be used as vaccine, for example for the prevention and therapeutic treatment of MUC1-positive carcinomas such as breast, colorectal, pancreatic and gastric cancer.

### BRIEF DESCRIPTION OF THE DRAWINGS

FIG. 1: MUC repeat peptide processing by human dendritic cells. Soluble antigen, a 100mer peptide with free amino and carboxy termini and corresponding to five repeats of the MUC1 repeat domain (HGV100), was used for pulsing of human immature dendritic cells prepared from peripheral blood monocytes. During pulsing the cells were simultaneously matured by induction with TNFα and anti CD40. After 24 h pulsing and maturation the cell supernatant was run over a solid-phase extraction column to isolate the peptide fragments. MALDI mass spectrometry in the positive ion mode revealed the formation of SAP17, GVT20, GVT23, and STA27 as the major cleavage products in the mass range from 1 to 3 kDa. Mass signal indicated by * represent peptide background not related to MUC1 antigen peptide.

FIG. 2: MUC1 glycopeptide processing by mouse dendritic cells. Bead-conjugated antigens, a mixture of biotinylated glycopeptides H1 to H3, SEQ ID NO: 5, (AHGVTSAPDTRPAPGSTAPPA) and H4 to H6 (AHGVTSAPESRPAPGSTAPAA), SEQ ID NO: 6, corresponding to a partial sequence of the MUC1 tandem repeat domain and glycosylated with GalNAc at Thr5 (H1, H4), Thr10/Ser10 (H2, H5) or Thr17 (H3, H6), was used for pulsing of mouse dendritic cells DC2.4. Processing products were affinity-isolated from cellular fractions or from culture supernatants by binding to streptavidin/polystyrene-coated beads, reduced with dithiothreitol to cleave the biotin label, and analysed by reflectron MALDI mass spectrometry in the positive ion mode. A, cellular fraction; B, cell culture supernatant; C, interpretation of mass spectrometric data. The major signals at m/z 2249.0 (H1 to H3), SEQ ID NO: 5, and 2223.0 (H4 to H6), SEQ ID NO: 6, correspond to the thiopropylated precursor glycopeptides, the signals at m/z 1695.7 (P1; SEQ ID NO: 7) and 1669.7 (P2; SEQ ID NO: 8) to the SAP16 fragments (P1 derived from H1 to H3; P2 from H4 to H6), which bind non-specifically to the polystyrene-coated bead surface.

FIG. 3: Peptide sequencing of processing products P1 and P2 by LC-MS/MS analysis on a Qtof2 electrospray mass spectrometer. Processing products in cellular supernatants from antigen-pulsed mouse DCs were separated by nanoflow liquid chromatography on a reversed-phase microcapillary column and analysed online by electrospray mass spectrometry in the positive ion mode. B-ion and y-ion fragment series from the N-terminal and C-terminal sequences of the major peptide products from endopeptidase cleavage were assigned after deconvolution of the spectrum (A; P1 at m/z 1695; B, P2 at m/z 1669) and were used to confirm the sequence of SAP16 glycopeptides derived from N-biotinylated H1 to H6 glycopeptide antigens (refer to C).

FIG. 4: In vitro proteolysis of MUC1 glycopeptide A3 by human cathepsin L. N-terminally free or biotinylated MUC1 glycopeptide A3 (10 μg) were treated for 3 h with 1 milliunit of cathepsin L in the presence or absence of the cathepsin L/B-specific cysteine protease inhibitor Z-Leu-Leu-Leu-fluoromethyl ketone (1 μM) using 0.1M sodium acetate, pH 5.5, containing 1 mM EDTA, and 1 mM DTT as reaction buffer.

Reflectron MALDI mass spectra were recorded in the positive ion mode using α-cyano-4-hydroxycinnamic acid as matrix. A, N-terminally free glycopeptide A3 in the absence of protease inhibitor (m/z 1857.7: SAP16; m/z 2324.0: A3 glycopeptide; Signals at m/z 1958.8 and 2115.8 correspond to products of a aminopeptidase contained in the human cathepsin L preparation); B, N-terminally free glycopeptide A3 in the presence of protease inhibitor; C, glycopeptide A3 N-terminally biotinylated with biotin N-hydroxysuccinimide ester (Sigma) at the amino terminus to block aminopeptidase activity (in the absence of protease inhibitor); (m/z 1858.6: SAP16; m/z 2549.8: biotinylated A3 glycopeptide); D, glycopeptide A3 N-terminally biotinylated with biotin N-hydroxysuccinimide ester at the amino terminus (in the presence of protease inhibitor).

FIG. 5: Cathepsin L-like activity in low-density endosomes from mouse dendritic cells cleaves MUC1 repeats at Thr-Ser: Low-density endosomes in mouse dendritic cells were separated from lysosomes and plasma membranes by density gradient centrifigation in percoll/sucrose (30 ml).

A profile of β-hexosaminidase activity in the gradient fractions demonstrates colocalisation of the lysosomal marker enzyme in high density fractions. The insert shows identification of cathepsin L in a westernblot of gradient fractions and human cathepsin L as a positive control. Fractions of 1 ml were collected and 20 μl samples were loaded onto 7.5% polyacrylamid gels. After SDS gelelectrophoresis the proteins were blotted onto nitrocelulose membranes and stained for the presence of cathepsin L using the monoclonal mouse antibody CPLH 3G10 defining a C-terminal peptide of murine and human mature enzyme (Alexis Deutschland, Grünberg, Germany).

FIG. 6: Proposed pathways of the cathepsin L-mediated processing of MUC1 tandem repeat peptide and its control by O-glycosylation. Filled arrows indicate cleavage sites of cathepsin L. Thin arrows indicate the formation of major (continuous lines) or minor fragmentation routes (dashed lines). GalNAc residues are marked by grey shaded rhombs, Gal residues by open circles.

### DETAILED DESCRIPTION OF THE INVENTION

The present invention relates to immunogenic MUC1 peptides, which can be used for immunization in mammals, especially in humans. In particular, those peptides are convenient in size, i.e. they comprise or consist of at least 9 consecutive amino acids derived from the tandem repeat domain of MUC1 and having the amino acid sequence SAP at their N-terminus.

The present invention is based on the observation that cathepsin L or a closely related enzyme shows a very restricted fragmentation pattern during human and mouse DC processing with only two preferred cleavage site per MUC1 repeat. Without intending to be bound by theory it is believed that the cleavage specificity and specific inhibition of the protease were in agreement with the assumption that cathepsin L or a closely related enzyme (cathepsins B or S) were involved in this highly specific cleavage.

The experimental set-up used biotinylated and non-tagged beads, coated with synthetic glycopeptides comprising one or more repeat units of MUC with single or multiple O-linked core-type glycans. Exogenously administered MUC1 peptide fragments were rapidly taken up by mouse dendritic cells (DCs) and a large proportion was processed in late endosomal compartments within 4 h. MUC1 repeat peptide derived proteolytic fragments that were identified and sequenced show that the glycans are not removed during antigen processing and that the presence of carbohydrates affects the cleavage sites yielding a different repertoire of cleaved peptides.

Surprisingly, the proteolytic products suggest a highly specific processing of the repeat peptide with one preferential cleavage site at the Thr-Ser peptide bond. While human cathepsin D was unable to cleave the MUC1 repeat peptide in vitro, human cathepsin L digestion resulted in specific hydrolysis of the Thr-Ser peptide bond. Since MUC1 sequences contain a VTSA motif in every repeat unit, the generated fragments start with the amino acid sequence SAP at their N-terminus. Furthermore, it turned out that cathepsin L cleaves the MUC1 repeat peptide at an additional site, namely at His-Gly. Thus, intermediate products arise from the processing of GVT-20 fragments (see for example SEQ ID NO: 12) that are transformed into SAP17 fragments by a further proteolytic cleavage depending on the site-specific O-glycosylation.

Information on the structure of processed MUC1 glycopeptides is of utmost importance for the design of tumor vaccines. Intact O-glycosylation on processed MUC1 repeat peptide contributes to a greater variety of the MHC class II-restricted helper T cell responses, thereby enhancing an overall anti-tumor response.

Hence, according to the invention, a peptide of least 9 amino acids in length derived from the tandem repeat domain of MUC1 and having the amino acid sequence SAP at its N-terminus is provided.

The amino and nucleic acid sequences of human MUC1 are known and can be found, for example, in the SWISS PROT and GenBank database; see, e.g., accession nos. NP_{—}877418 and NM_{—}182741.1 and references cited therein. The MUC1 protein contains varying numbers of amino acids due to a length polymorphism resulting from individually variable repeat numbers, and, in the moment, at least 9 isoforms are known (1/A, 2/B, 3/C, 4/D, 5/SEC, 6/X, 7/Y, 8/Z and 9/S, which are produced by alternative splicing).

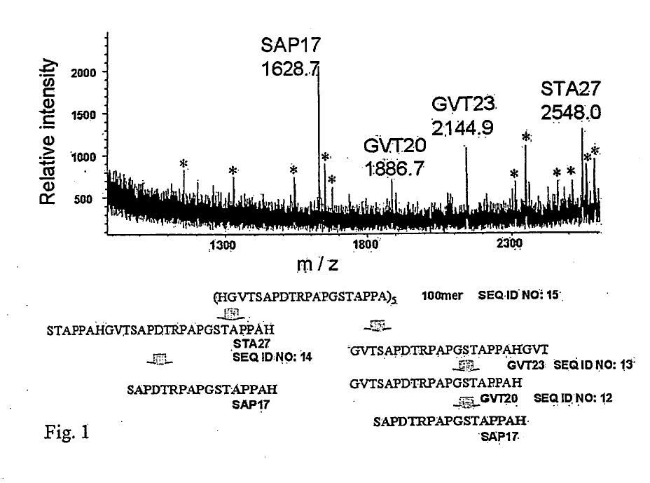

In this invention, specific peptides of MUC1 are contemplated, which are derived from a synthetic or natural MUC1 sequence, which has been cleaved enzymatically at the VTSA motif contained in all MUC1 sequences (or was chemically synthesized in case of synthetic fragments). In one embodiment, the peptides of the present invention thus can be obtained by cleavage of MUC1 sequences with cathepsin-L. Irrespective of the starting amino acid position in the repeat sequence (TAP, AHG, GST) and of the length of the peptides (20mer, 21mer, 25mer, 100mer), cathepsin L cleaves specifically between Thr-Ser in the VTSA motif of the repeat peptide, thereby resulting in the peptides according to the invention. It is an essential feature of the present invention that all peptides have the amino acid sequence SAP at or near their N-terminus. The most important feature of the peptides of the invention is that they consist of or comprise at least one tandem repeat domain of at least 9 amino acids as shown below for the peptides of SEQ ID NOS: 1 to 4 and 11, with a minimum tandem repeat sequence of 9 amino acids, e.g. from position 1 to 9 of any one of SEQ ID NOS: 1 to 4 and 11. This also means that the amino acid sequence SAP does not need to be immediately at the N-terminus but may be preceded by one or more amino acids, for example with the amino acid sequence GVT with or without an additional amino acid such as H, see, e.g., peptide fragments shown in FIGS. 1 and 6. However, peptides consisting or comprising said tandem repeat domain with N-terminal deletions of one or more amino acids, even of the SAP motif, are encompassed in the scope of the present invention as well, in particular if those peptide variants exhibit substantially the same immunological and/or biological activity as a reference peptide such as SAP 17.

As mentioned above, the peptide of the present invention is not limited in its length, and may, for example, comprise up to 100 amino acids or even more. Irrespective the theory behind the molecular mechanism of action, the peptides of the invention have at least 9 preferably 10, more preferably 12, still more preferably 15 or 20, and most preferably 10 to 25 or 30 consecutive amino acids derived from said tandem repeat, and wherein said peptides are capable of evoking an immune response in a mammal, in particular humans; see also the examples. Since cathepsin L, as mentioned above, furthermore is in the position for a proteolysis at His-Gly particularly peptides with 17 amino acids are generated according to the invention (i.e. the MUC1 repeat peptide is cleaved at two sites in one repeat unit (namely at Thr-Ser and His-Gly) which results in a fragment of 17 amino acids, see also FIG. 6).

The degradation down to the level of SAP17, however, is inhibited by O-glycosylation at Thr or Ser within the VTSA motif, so that respectively glycosylated GVT20 peptides are generated as final products, in particular if O-glycosylation is substantially restricted to GalNac-residues while longer glycan chains may interfere with processing mediated by cathepsin L. In view of the naturally occurring peptides SAP17 and GVT20, peptides of 17 to 20 amino acids in length are particularly preferred.

In one embodiment, the peptide according to the invention is a fragment of said tandem repeat domain. Such fragment can be derived from the tandem repeat domain for example by cleavage with cathepsin L or (an) other enzyme(s) resulting in a peptide according to the invention; see also infra and the examples.

According to a further embodiment, the invention provides specific peptides which comprise an amino acid of any one of SEQ ID NOS: 1 to 4 or 11, or variants thereof, wherein said variants may comprise one or more amino acid additions, insertions, substitutions and/or deletions as compared to the sequence of SEQ ID NOS: 1 to 4 or 11, and wherein the biological activity, i.e. immunological activity is substantially the same as the activity of the peptide comprising the unmodified amino acid sequence of SEQ ID NOS: 1 to 4 or 11. In this context, the present invention provides the following peptides:

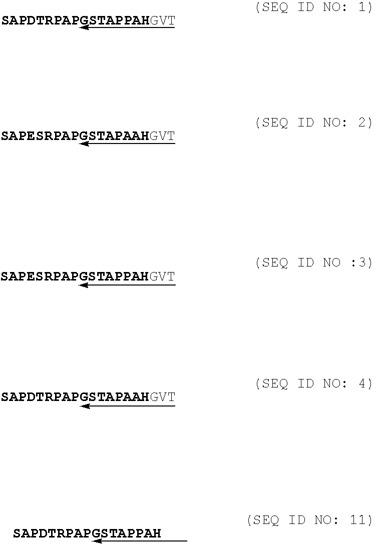

The arrow indicates that the present invention also encompasses variants of the above mentioned amino acid sequences, which are reduced by one or more amino acids starting from the C-terminus, under the proviso that the variants at least comprise the 9 N-terminal amino acids of the above indicated sequences (printed in bold).

The peptides of the present invention can be in their free acid form or they can be amidated at the C-terminal carboxylate group. The present invention also includes analogs of the peptides of the invention. An “analog” of a polypeptide includes at least a portion of the polypeptide, wherein the portion contains deletions or additions of one or more contiguous or noncontiguous amino acids, or containing one or more amino acid substitutions.

“Insertions” or “deletions” are typically in the range of about 1 to 3 amino acids. The variation allowed may be experimentally determined by systematically making insertions, deletions, or substitutions of amino acids in a polypeptide molecule using recombinant DNA techniques and assaying the resulting recombinant variants for activity. This does not require more than routine experiments for the skilled artisan. In case of MUC1 repeats three positions are known to exhibit a sequence polymorphism in the population (Engelmann et al., J. Biol. Chem. 276 (2001), 27764-27769; international patent application WO00/49045, the disclosure of which is incorporated in its entirety in this application by reference).

Substitutes for an amino acid in the polypeptides of the invention are preferably conservative substitutions, which are selected from other members of the class to which the amino acid belongs. An analog can also be a larger peptide that incorporates the peptides described herein. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For the purposes of this invention, conservative amino acid substitutions are defined to result from exchange of amino acids residues from within one of the following classes of residues: Class I Ala, Gly, Ser, Thr, and Pro; Class II: Cys, Ser, Thr, and Tyr; Class III: Glu, Asp, Asn, and Gln (carboxyl group containing side chains): Class IV: His, Arg, and Lys (representing basic side chains); Class V: Ile, Val, Leu, Phe, and Met (representing hydrophobic side chains); and Class VI: Phe, Trp, Tyr, and His (representing aromatic side chains). The classes also include other related amino acids such as halogenated tyrosines in Class VI.

Peptide analogs, as that term is used herein, also include modified peptides. Modifications of peptides of the invention include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

The peptide of the present invention may also comprise one of the group of D-isomer amino acids, L-isomer amino acids, or a combination thereof. The preparation of peptides comprising D-isomer amino acids is described for example in Schumacher, Science 271 (1996), 1854-1857.

The term “biological activity” as used herein is related to the immunogenic function of the amino acid sequences according to the invention. As mentioned above, MUC1 is naturally overexpressed in various cancers, like breast cancer and other adenocarcinomas, and therefore, it is an important target for immune based anti-cancer therapy. Thus, the MUC1 peptides as disclosed hereinbefore are contemplated as long as they are capable of inducing an immungenic reaction in mammals, preferably humans, in order to initiate/promote an attack of the patient's immune system against the respective cancer.

The present invention is further directed to a nucleic acid encoding one of the above mentioned peptides. The term “nucleic acid”, “nucleic acid sequence” and “polynucleotide” are used interchangeably herein and refer to a heteropolymer of nucleotides or the sequence of these nucleotides. The polynucleotides of the present invention also include, but are not limited to, polynucleotides that hybridize to the complement of the disclosed nucleotide sequences under moderately stringent or stringent hybridization conditions; a polynucleotide which is an allelic variant of any polynucleotide recited above; a polynucleotide which encodes a species homologue of any of the herein disclosed proteins; or a polynucleotide that encodes a polypeptide comprising an additional specific domain or truncation of the disclosed proteins. Stringency of hybridization, as used herein, refers to conditions under which polynucleotide duplexes are stable. As known to those of skill in the art, the stability of duplex is a function of sodium ion concentration and temperature (see, for example, Sambrook et al., Molecular Cloning: A Laboratory Manual 2^{nd} Ed. (Cold Spring Harbor Laboratory, (1989)). Stringency levels used to hybridize can be readily varied by those of skill in the art.

Low stringency hybridization refers to conditions equivalent to hybridization in 10% formamide, 5× Denhart's solution, 6×SSPE, 0.2% SDS at 42° C., followed by washing in 1×SSPE, 0.2% SDS, at 50° C. Denhart's solution and SSPE are well known to those of skill in the art as are other suitable hybridization buffers.

Moderately stringent hybridization refers to conditions that permit DNA to bind a complementary nucleic acid that has about 60% identity, preferably about 75% identity, more preferably about 85% identity to the DNA; with greater than about 90% identity to said DNA being especially preferred. Preferably, moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 65° C.

High stringency hybridization refers to conditions that permit hybridization of only those nucleic acid sequences that form stable duplex in 0.018M NaCl at 65° C. (i.e., if a duplex is not stable in 0.01 SM NaCl at 65° C., it will not be stable under high stringency conditions, as contemplated herein).

Further, nucleic acid hybridization techniques can be used to identify and obtain a nucleic acid within the scope of the invention. Briefly, any nucleic acid having some homology to a sequence set forth in this invention, or fragment thereof, can be used as a probe to identify a similar nucleic acid by hybridization under conditions of moderate to high stringency. Such similar nucleic acid then can be isolated, sequenced, and analyzed to determine whether they are within the scope of the invention as described herein.

According to a preferred embodiment, the peptides of the present invention are O-glycosylated at one or more of the threonines or serines contained in the sequence. Preferably, the peptides of any one of SEQ ID NOS: 1 to 4 or 11 are glycosylated at Thr 5 and/or 12. However, also all other serines or threonins in the respective sequences may be glycosylated. A preferred glycan used herein is GalNAc or further complex glycans, which are derived therefrom.

According to a further aspect, the present invention provides a method of producing the peptides according to the invention, comprising the following steps:
(a) providing a peptide comprising the tandem repeat domain of MUC1 or a part thereof, which part at least contains one repeating unit of said tandem repeat domain of MUC1;
(b) contacting the peptide of (a) with an effective amount of cathepsin-L or a closely related enzyme hereof, thereby cleaving the peptide; and
(c) isolating the fragments produced in (b).

Preferably, the peptide provided in (a) is a MUC1 protein showing a natural glycosylation pattern. As mentioned above, it was surprisingly found in accordance with the present invention that a cathepsin-L cleavage as performed in step (b), leaves the glycosylation pattern of the MUC1 protein, provided in (a), intact. Intact O-glycosylation on processed MUC1 repeat peptides in turn contributes to a greater variety of the MHC class II-restricted helper T cell responses, thereby enhancing an overall anti-tumor response in patients. Thus, the method of the invention leads to a MUC1 peptide, which can be easily processed by the patient's APCs, for example dendritic cells, by the MHC class II pathway, and will be presented with an intact glycosylation pattern leading to an enhanced immune response of helper T-cells. In this context, it should be noted that there is no restriction regarding the glycosylation pattern, however, threonin glycosylated at the cleavage site leads to a Thr-Ser bonding, which is stable to cathepsin L proteolysis. Glycosylation at other sites does not disturb the cleavage according to the invention by cathepsin L, but a multiple Gal-GalNAc-substitution as well as a substitution with complex glycans may hamper or even inhibit a fragmentation at His-Gly.

Of course, the above mentioned method is not the only one which leads to said peptides, whether glycosylated or not. It is also possible to chemically synthesize those peptides thereby providing, for example, a desired glycosylation pattern. To synthesize glycopeptides, glycosylamino acid building blocks are required which already contain the oligosaccharide chain and threonine or serine. The syntheses of these building blocks have been described (Mathieux et al., J. Chem. Soc., Perkin Trans. 1 (1997), 2359-2368). The multiple column solid phase synthesis can be carried out in a semi-manual 20-column multiple synthesizer, and Wang resin can be selected as support material. The Wang resin (2.5 g) can for example be placed in a glass reactor, swelled in dichloromethane (15 cm³, 10 min.) and washed. A mixture of Fmoc-Ala-OH (3,40 mmol), 1-(mesitylenesulfonyl)-3-nitro-1,2,4-triazole (3,40 mmol) and methylimidazole (3,40 mmol) in dichloromethane (15 cm³) was added. After 2 h, the resin can be washed and the unchanged amino groups can be acetylated with Ac₂O/DMF (1:1; 15 cm³). The derivatized resin is then packed for the glycopeptide synthesis in the 20 columns of the synthesizer. The reaction and washing solvent can be DMF, the Fmoc deprotections were performed by treatment with piperidine (20%) in DMF (20 min.). The amino acids are coupled as Fmoc amino acid Pfp ester with Dhbt-OH (3 mol equiv.). The Gal(1→3)GalNAc-containing building block are coupled with TBTU and N-ethyldiisopropylamine (1.5 mol equiv.). After 20 h reaction time the synthesis cycle is repeated to complete the assembly of each glycopeptide. After removal of the last Fmoc groups, the resins are washed, dried, treated with 95% aq TFA (2 cm³, 2 h), and filtered off. Then, the compounds is treated with catalytic amounts of 1% CH₃ONa in methanol at pH 8.5 to remove the acetylic groups of the saccharide part, and purified by preparative RP-HPLC. The pure O-glycopeptides are obtained in yields of 16-57% after lyophilization. Preferably, glycopeptides are formed containing O-linked GalNAc or elongated complex glycans at one or several of the threonine or serine residues.

The peptides of the invention may also be synthesized by the solid phase method using standard methods based on either t-butyloxycarbonyl (BOC) or 9 fluorenylmethoxy-carbonyl (FMOC) protecting groups. This methodology is described by G. B. Fields et al. in Synthetic Peptides: A User's Guide, W. M. Freeman & Company, New York, N.Y. (1992), 77-183. The present peptides may also be synthesized via recombinant techniques well known to those skilled in the art. For example, U.S. Pat. No. 5,595,887 describes methods of forming a variety of relatively small peptides through expression of a recombinant gene construct coding for a fusion protein which includes a binding protein and one or more copies of the desired target peptide. After expression, the fusion protein is isolated and cleaved using chemical and/or enzymatic methods to produce the desired target peptide.

Preferably, the peptide provided in step (a) is represented by natural MUC1 derived from human milk fat membranes (see Müller et al., J. Biol. Chem. 272 1997, 24780-24793), from tumor ascites (Beatty et al., Clin. Cancer Res. 7 (2001), 781-787) or from human breast carcinoma cell lines (Müller et al., J. Biol. Chem. 277 (2002), 26103-26112) or is represented by any one of SEQ ID NOS: 5, 6, 9 or 10 or 12.

Furthermore, the amino acids of the peptide provided in step (a) of the above method of producing the peptides of the invention are O-glycosylated, however, provided that the peptide is not glycosylated at the cleaving site of cathepsin-L. Preferably, one or more of the threonines or serines of the peptide isolated in (c) are O-glycosylated.

According to a further aspect, a peptide is provided, which is obtainable by the above mentioned methods. The peptides of the present invention may be employed in a monovalent state (e.g., free peptide or peptide coupled to a carrier molecule or structure).

The peptides may also be employed as conjugates having more than one (same or different) peptide bound to a single carrier molecule. The carrier molecule or structure may be microbeads, liposomes, biological carrier molecule (e.g., a glycosaminoglycan, a proteoglycan, albumin, or the like), a synthetic polymer (e.g., a polyalkyleneglycol or a synthetic chromatography support), biomaterial (e.g., a material suitable for implantation into a mammal or for contact with biological fluids as in an extracorporeal device), or others. Typically, ovalbumin, human serum albumin, other proteins, polyethylene glycol, or the like are employed as the carrier. Such modifications may increase the apparent affinity and/or change the stability of a peptide. The number of peptide fragments associated with or bound to each carrier can vary. In addition, as mentioned above, the use of various mixtures and densities of the peptides described herein may allow the production of complexes that have specific binding patterns in terms of preferred ligands.

The peptides can be conjugated to other peptides using standard methods known to one of skill in the art. Conjugates can be separated from free peptide through the use of gel filtration column chromatography or other methods known in the art.

For instance, peptide conjugates may be prepared by treating a mixture of peptides and carrier molecules (or structures) with a coupling agent, such as a carbodiimide. The coupling agent may activate a carboxyl group on either the peptide or the carrier molecule (or structure) so that the carboxyl group can react with a nucleophile (e.g., an amino or hydroxyl group) on the other member of the peptide conjugate, resulting in the covalent linkage of the peptide and the carrier molecule (or structure).

As another example, peptides may be coupled to biotin-labeled polyethylene glycol and then coupled to avidin containing compounds. In the case of peptides coupled to other entities, it should be understood that the designed activity may depend on which end of the peptide is coupled to the entity.

Accordingly, in another aspect the present invention relates to a fusion molecules, also referred to herein as peptide conjugates, comprising a peptide of the invention.

The invention is further directed to an ex vivo-method of producing a population of autologous antigen presenting cells (APCs), which are capable of inducing effective immune responses against MUC1, comprising the steps of
(a) providing autologous APCs from a tumor patient;
(b) contacting the autologous APCs from the tumor patient with an effective amount of a peptide or fusion molecule of the invention under conditions which allow endocytosis, processing and MHC class II presentation of the peptides by said APCs; and
(c) isolating said peptide presenting APCs for the purpose of immunotherapeutic application in the patient.

Preferably, the MUC1 peptides in (a) are bound to coated ferric oxide beads. However, it is noted that all other known beads or other carriers and/or conjugates known in the art can be used for the purpose of the above mentioned method. Generally, all beads can be used, which are not larger than approx. 1-2 μm in size and allow a covalent coupling of antibodies and lectines.

Furthermore, an ex vivo-method of producing genetically engineered APCs is provided, which are capable of inducing effective immune responses against MUC1, comprising the steps of:
(a) providing a nucleic acid, which encodes one of the peptides or the fusion molecule of the invention;
(b) transfecting the APCs with said nucleic acid; and
(c) selecting APCs, which present said peptides in an MHC II restricted manner.

According to a preferred embodiment, the nucleic acid in step (a) is provided in an expression vector. This expression vector preferably comprises one or more regulatory sequences. The term “expression vector” generally refers to a plasmid or phage or virus or vector, for expressing a polypeptide from a DNA (RNA) sequence. An expression vector can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an N-terminal methionine residue. This residue may or may not be subsequently cleaved from the expressed recombinant protein to provide a final product.

According to a further aspect of the invention, an APC is provided, which is obtainable by one of the aforementioned methods. Preferably, this APC is a dendritic cell or a B cell.

Furthermore, the present invention provides a therapeutic or pharmaceutical composition, comprising the peptide, nucleic acids, vectors, fusion molecule and/or the APCs of the invention and a pharmaceutically acceptable carrier. Such a composition may also contain (in addition to the ingredient and the carrier) diluents, fillers, salts, buffers, stabilizers, solubilizers and other materials well known in the art. The term “pharmaceutically acceptable” means a non-toxic material that does not interfere with the effectiveness of the biological activity of the active ingredient(s). The characteristics of the carrier will depend on the route of administration. The therapeutic composition may further contain other agents which either enhance the activity or use in treatment. Such additional factors and/or agents may be included in the therapeutic composition to produce a synergistic effect or to minimize side-effects. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients.

Techniques for formulation and administration of the compounds of the present application may be found in “Remington's Pharmaceutical Sciences”, Mack Publishing Co., Easton, Pa., latest edition.

The compositions contain a therapeutically effective dose of the respective ingredient. A therapeutically effective dose refers to that amount of the compound sufficient to result in amelioration of symptoms, e.g., treatment, healing, prevention or amelioration of such conditions, specifically in an induction of an immune response in the patient. Suitable routes of administration may, for example, include parenteral delivery, including intramuscular and subcutaneous injections, as well as intrathecal, direct intraventricular, intravenous, intraperitoneal injections. Intravenous administration to the patient is preferred.

A typical composition for intravenous infusion can be made up to contain 250 ml of sterile Ringer's solution, and 10 mg of the ingredient; see Remington's Pharmaceutical Science (15^{th} Ed., Mack Publishing Company, Easton, Pa., 1980). Preferably, the therapeutic composition of the present invention is a vaccine. As mentioned above, this vaccine finds application for use in the treatment of breast cancer or other MUC1-positive carcinomas including colorectal, pancreatic and gastric carcinomas.

The present invention is furthermore directed to the use of the peptides, the nucleic acids, the fusion molecule and/or the APC's of according to the invention for the preparation of a pharmaceutical composition for the treatment of MUC1-positive carcinomas. These carcinoma include breast, colorectal, pancreatic and gastric cancer as mentioned herein before. The agents of the present invention are preferably formulated in pharmaceutical compositions and then administered to a patient, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, intraorgan, intraarterial and intravenous) administration.

Formulations suitable for parenteral administration conveniently include a sterile aqueous preparation of the active agent, or dispersions of sterile powders of the active agent, which are preferably isotonic with the blood of the recipient. Absorption of the active agents over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active agent as a powder, or granules, as liposomes containing the active agent, or as a solution or suspension in an aqueous liquor or non-aqueous liquid such as a syrup, an elixir, an emulsion, or a draught. Such compositions and preparations typically contain at least about 0.1 wt-% of the active agent. The amount of peptide (i.e., active agent) is such that the dosage level will be effective to produce the desired result in the patient.

Aerosol formulations such as nasal spray formulations include purified aqueous or other solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier.

In addition, the invention relates to a method of treatment of patients suffering from a MUC1-positive carcinoma, wherein the therapeutic composition described above is administered to the patient in an amount effective to induce an immune response against MUC1. The appropriate concentration of the therapeutic agent might be dependent on the particular agent. The therapeutically effective dose has to be compared with the toxic concentrations; the clearance rate as well as the metabolic products play a role as do the solubility and the formulation. Therapeutic efficacy and toxicity of compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

Unless otherwise defined, all technical and scientific terms used herein have the same meaning as commonly understood by one of ordinary skill in the art to which this invention pertains. The above disclosure generally describes the present invention. Several documents are cited throughout the text of this specification. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention. In case of conflict, the present specification, including definitions, will control.

The above disclosure generally describes the present invention. A more complete understanding can be obtained by reference to the following specific examples intended to limit the scope of the invention.

### EXAMPLES

The examples which follow further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003).

The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11, (2001), 98-107.

### Example 1

### Processing of MUC1 by Human Dendritic Cells is Site-Specific

Isolation and Cultivation of Dendritic Cells

Peripheral blood mononuclear cells (PBMCs) from healthy donors were obtained by leukapheresis followed by Ficoll-density centrifugation. CD14⁺ cells were positively selected using CD14-Microbeads and MACS separation (Miltenyi Biotech, Bergisch Gladbach, Germany) and subsequently cultured for 8 days in CellGro Medium (Cellgenix, Freiburg, Germany) supplemented with 800 U/ml of granulocyte-macrophage colony-stimulating factor (GM-CSF; Sandoz, Basel, Switzerland) and 500 IU/ml of IL-4 (CellGenix) at 37° C. and 5% CO₂. GM-CSF and IL-4 were replenished on days 3 and 5 of culture.

Immortalized dendritic cells (clone D2.4) from C57BL/6 mice were grown in DMEM supplemented with 10% FCS, L-glutamine, 0.1% 2-mercaptoethanol, and antibiotics at 37° C. and 5% CO₂ (Shen et al., J. Immunol. 158 (1997), 2723-2730).

MUC1 Glycoforms

Native MUC1 glycoforms were isolated from human tumor ascites (Beatty et al., Clin. Cancer Res. 7 (2001), 781-787) or from human milk fat globule membranes as described previously. A partially deglycosylated derivative of the lactation-associated glycoform was generated by treatment with trifluoromethane sulfonic acid for 30 min at 0° C. (Müller et al., J. Biol. Chem. 272 (1997), 24780-24793). Recombinant fusion protein containing six MUC1 repeats was isolated from the cell culture supernatants after expression in the embryonic kidney cell line EBNA-293 as described earlier (Müller and Hanisch, J. Biol. Chem. 277 (2002), 26103-26112).

Generation of Synthetic MUC1 Glycopeptides

Glycopeptides H1 to H6 corresponding to MUC1 tandem repeat peptides based on the AHG21 sequences AHGVTSAPDTRPAPGSTAPPA (H1 to H3) and AHGVTSAPESR PAPGSTAPAA (H4 to H6) and carrying GalNAc at Thr5, Thr10, or Thr17 were chemically synthesized-according to previously-published protocols (Karsten et al., Cancer Res. 58 (1998), 2541-2549) and kindly provided by Prof. Hans Paulsen (Institute of Organic Chemistry, University of Hamburg, Germany). The same holds true for glycopeptide A3 (substituted with Galβ1-3GalNAc at Thr17), which is based on the same peptide sequence as H1 to H3. The 100mer peptide corresponding to five repeats of the MUC1 domain and starting with the HGV motif was synthesized by a local facility (University of Pittsburgh) and in vitro glycosylated with GalNAc using purified polypeptide GalNAc-transferases-T1 and -T2 (kindly provided by Dr. Henrik Clausen, School of Dentistry, University of Copenhagen, Denmark) under conditions described previously (Hanisch et al., J. Biol. Chem. 274 (1999), 9946-9954; Hanisch et al., Glycobiology 11 (2001), 731-740). TAP25 and GST20-AES were synthesized in a local facility at the Institute of Biochemistry (Cologne, Germany).

Antigen Pulse of Dendritic Cells

Human immature DCs were pulsed with native, N-terminally unmodified, soluble antigens, while the mature mouse DCs had to be fed with particulate antigen to reach sufficient antigen load. Human monocyte-derived immature DCs (10⁷ cells in 5 ml Cellgro medium) were pulsed in 6-well cell-culture plates Munc, Wiesbaden, Germany) by incubation with 20 μg/ml soluble antigen (native mucin from tumor ascites, 100mer peptide) for a period of 24 h. Simultaneously, the maturation process of the cells was induced by addition of 20 ng/ml tumor necrosis factor (TNF-α; Sigma-Aldrich, Munich, Germany) and 10 μg/ml of anti-CD40 antibody (Pharmingen, San Diego, Calif.). The cells were finally separated from medium by centrifugation, washed in PBS and both fractions were analysed for the presence of peptide/glycopeptide fragments according to the protocol described below.

Mouse dendritic cells DC2.4 (10⁷ cells/1 to 10 ml) were transferred into a 15 ml Falcon tube, suspended in AIMV medium and preincubated for 1 h at 37° C. (5% CO₂). Antigens were added as native MUC1 (100 μg from tumor ascites or milk fat globule membranes), as recombinant fusion protein (100 μg), a 100mer repeat peptide (100 μg) or as a mixture of biotinylated glycopeptides H1 to H6 (50 μg) after conjugation to anti-MUC1 antibody (B27.29)-coated dynabeads (each at 5×10⁷ beads/ml final concentration). The 1 ml suspension was incubated with occasional shaking at 37° C. (5% CO₂) for a total time period of 4 h. After pulsing the cells were separated from the medium by centrifugation (180 g, 5 min). The cell fraction was washed several times in phosphate (4 mM), NaCl (153 mM), pH 7.2, while the cell-free supernatant was re-centrifuged at 3000 g (5 min, 4° C.).

Isolation of Peptides

The human or mouse dendritic cell fractions were treated on ice for 15 min with 100 μl 1% NP40, 10 mM Tris-HCl, 150 mM NaCl, pH 8.0 containing a cocktail of protease inhibitors (Sigma P8340, München, Germany) followed by ultrasonication for 2 min. Isolation of MUC1-derived (glyco)peptides was performed in parallel alternative ways: 1) by affinity chromatography on anti-MUC1 (BW835, C595) antibody columns; 2) by solid-phase extraction on polysphere C18 columns or on Poros 20 R2 beads (PerSeptive Biosystems, Framingham, USA), and 3) by binding to streptavidin-coated magnetic beads (Dynal). To avoid selection of product subfractions by affinity-isolation, in most experiments the peptides were enriched by solid-phase extraction on reversed-phase columns. Due to high antigen load of the cells this non-selective enrichment of peptides was sufficient to detect MUC1-specific proteolysis products by mass spectrometry in the presence of cellular background.

Anti-MUC1 columns with a total of 1 mg immobilized antibodies were prepared using HighTrap-NHS columns from Amersham-Pharmacia according to the manufacturer's instructions. Antibody BW835 was kindly supplied by Behring-Werke, (Marburg, Germany). The cell extracts were cycled twice over the PBS equilibrated columns at a flow rate of 6 ml per hour in the cold and bound peptides were eluted with 0.1% TFA. Alternatively, the cell extracts were diluted twofold with PBS and incubated with 2×10⁸ streptavidin-coated dynabeads M-270 for 30 min at 37° C. and another 30 min period with rolling at ambient temperature. After magnetic separation and washing of the beads for three times the beads were treated with 10 mM dithiothreitol at 56° C. (30 min), and the dried eluate was taken up in 0.1% aqueous trifluoroacetic acid (TFA). Considerable amounts of non-tagged MUC1 glycopeptides were demonstrated to bind to streptavidin-polystyrene-coated dynabeads and to elute during heating under reducing conditions.

Peptides and glycopeptides contained in the cell-free supernatants were affinity-isolated by solid-phase extraction on reversed-phase supports (100 μg polysphere C18, 50 μl Poros C18).

After activation (80% ACN/0.1% TFA), equilibration (water) and loading of the reversed-phase column with 0.5 to 2 ml of supernatant, the sample was desalted by washing with water and eluted with 80% acetonitrile (ACN) in 0.1% aqueous TFA.

Mass Spectrometric Analyses

MALDI mass spectrometry: The peptide and glycopeptide samples (20 μl) contained in 0.1% aqueous TFA or in mixtures with acetonitrile were applied to the stainless steel target by mixing a 1 μl aliquot with the same volume of matrix (saturated solution of α-cyano-4-hydroxycinnamic acid in ACN/0.1% TFA, 2:1). Mass spectrometric analysis was performed on a Bruker-Reflex IV instrument (Bruker-Daltonic, Bremen, Germany) by positive ion detection in the reflectron mode. Ionization of co-cristallized analytes was induced with a pulsed nitrogen laser beam (337 nm) and the ions were accelerated in a field of 20 kV and reflected at 23 kV (Hanisch et al., Glycobiology 11 (2001), 731-740; Müller et al., J. Biol. Chem. 274 (1999), 18165-18172).

Nanoflow liquid chromatography with on-line ESI mass spectrometry: LC/MS data were acquired on a Q-Tof 2 quadrupole-time of flight mass spectrometer (Micromass, Manchester, UK) equipped with a Z spray source. Samples were introduced using the Ultimate nano-LC system (LC Packings, Amsterdam, Netherlands) equipped with the Famos autosampler and the Switchos column switching module. The column setup comprised a 0.3 mm×1 mm trap column and a 0.075×150 mm analytical column, both packed with 3 μm PepMap C18 (LC Packings, Amsterdam, Netherlands). Samples were diluted 1:10 in 0.1% TFA. 10 μl were injected onto the trap column and desalted for 3 min using 0.1% TFA and a flow rate of 30 μl/min. The 10 port valve switched the trap column into the analytical flowpath and the peptides were eluted onto the analytical column using a gradient of 5% ACN in 0.1% formic acid to 40% ACN in formic acid over 20 min and a column flow rate of approximately 200 nl/min, resulting from a 1:1000 split of the 200 μl/min flow delivered by the pump. Survey scans of 1 sec covered the range from m/z 400 to m/z 1200. Doubly and triply charged ions rising above a given threshold were selected for MS/MS experiments. In MS/MS mode the mass range from m/z 40 to m/z 1400 was scanned in 1 sec and 10 scans were added up for each experiment. Doubly and triply charged ion masses were deconvoluted using the MaxEnd software and the b- and y-ion series were assigned.

Human monocyte-derived immature DCs have previously been studied for their ability to take up soluble MUC1 peptide antigen by macropinocytosis and demonstrated to reach maximum levels of incorporation within 2 hours (Vlad et al., J. Exp. Med. 196 (2002), 1435-1446). Antigen uptake over a period of 24 h was not affected by parallel induction of the maturation process with TNFα and anti-CD40.

Human CD1a⁺ CD14⁻ CD83⁻ dendritic cells were pulsed with native mucin from tumor ascites or 100mer peptide either as soluble antigen or as antibody complex. The antibody C595 complex of 100mer peptide was not more efficiently incorporated and processed by the cells than free antigen according to quantitative HPLC measurement of 100mer peptide and derived proteolytic fragments in the culture supernatants. In case of 100mer peptide, a fraction of the antigen (below 5%) was processed and the proteolytic products were detected in the cell lysates as well as in the culture supernatants. Peptide fragments registered by positive ion MALDI(tof) mass spectrometry in the mass range from 1 to 3 kDa were detected at m/z 1628.7 (SAP17), 1886.7 (GVT20), 2144.9 (GVT23), and 2548.0 (STA27) (FIG. 1) and identified by LC-ESI-MS/MS (not shown). No fragmentation of antigen was revealed after pulsing of DCs with native MUC1 from tumor ascites according to mass spectrometric analyses of cellular lysate or culture supernatant in the mass range up to 8 kDa. All supernatants were checked for the absence of secreted cathepsin L-related activities by incubation of TAP25 peptide and A3 glycopeptide for 24 h at 37° C. and mass spectrometric analysis of solid-phase extracted fractions.

### Example 2

### Site-Specific Processing of MUC1 by Mouse Dendritic Cells is Controlled by O-Linked Glycans

Preparation of Bead-Coated Antigen

In some experiments, in which mature mouse DCs were pulsed, a selected panel of glycopeptides was non-covalently conjugated to antibody-coated beads. Glycopeptides H1 to H6 (100 μg each) were used unmodified or biotinylated with [2-(biotinamido) ethylamido]-3,3′-dithiopropionic acid N-hydroxysuccinimide ester (Sigma, München, Germany; 100 mM in DMSO, 100 μl) at 50° C. over a period of 48 h. After evaporation of the solvent by vacuum centrifugation the biotinylated products were separated from non-tagged glycopeptides and excessive reagent by reversed-phase chromatography on a PLRP-S column (Polymer Laboratories, Shropshire, UK). Anti-MUC1 dynabeads were prepared by covalent coupling of 50 μg B27.29 monoclonal antibody (Biomira, Edmonton, Canada) to tosyl-activated M-280 beads (Dynal, Hamburg, Germany) in 0.1 M borate buffer, pH 9.5 (200 μl) for 48 h at ambient temperature. Lectin-coated dynabeads were prepared similarly by conjugation of 50 μg Helix pomatia agglutinin to M-280 beads. Antibody- and lectin-coated beads (10⁸) were complexed with glycopeptides (50 μg) by incubation in 250 μl AIMV medium under rolling for 2 h at ambient temperature.

Confocal Laser Scanning Microscopy and Fluorescence-Activated Cell Sorting

Antigen uptake was quantitated by flow cytometric analysis using a Becton Dickinson FACScalibur according to a previously published protocol (Hiltbold et al., Cell. Immunol. 194: 143-149, 1999). Prior to microscopic inspection-DCs were fixed with 2% formaldehyde, and permeabilized with 0.1% saponin. Following staining with anti-MUC1 antibody (B27.29, Biomira, Edmonton, Canada), biotinylated secondary anti-mouse Ig (Dako, Hamburg, Germany) and FITC-labelled streptavidine (Sigma), the cells were fixed a second time with 1% paraformaldehyde, the chambers of the slides were removed, and the slides were mounted for the analysis by confocal laser scanning microscopy on a Leica DM IRE2 (Hiltbold et al., J. Immunol. 165 (2000), 3730-3741).

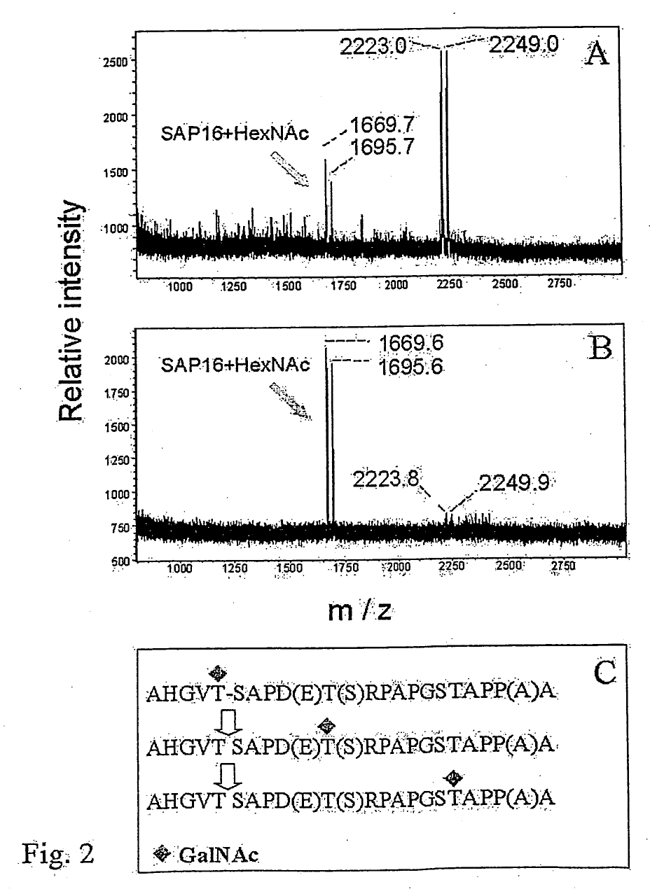

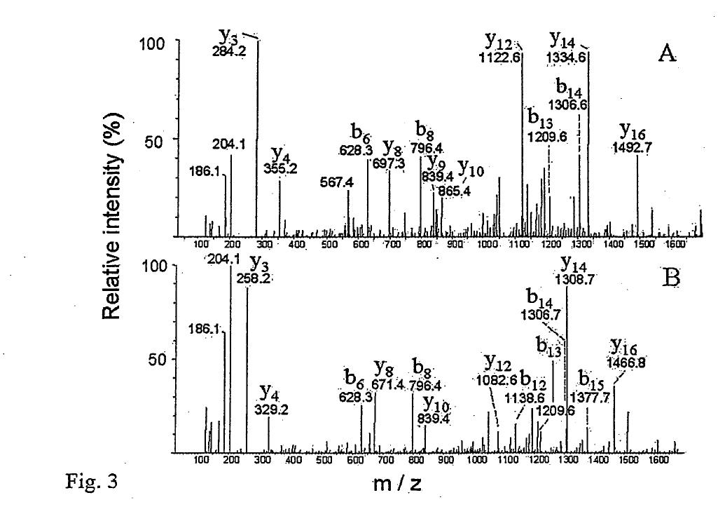

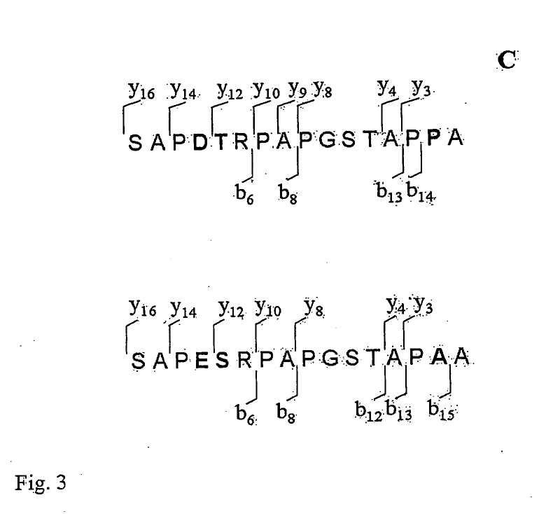

The mouse cell line DC2.4 representing mature dendritic cells is known to have low capacities for antigen uptake by macropinocytosis or receptor-mediated endocytosis, but has been reported to incorporate particle bound antigen very effectively (Shen et al., J. Immunol. 158 (1997), 2723-2730). For this reason, processing of MUC1 by mouse DCs was studied by using bead-conjugated antigen. Mouse DCs were pulsed with native MUC1 antigen, recombinant fusion protein, 100mer peptide or with a mixture of biotinylated glycopeptides (H1-H6) conjugated to antibody- and/or lectin-coated beads (Tab. 1). Two fractions, the cell pellet and the supernatant were analysed for the presence of proteolytic fragments by MALDI(tof) mass spectrometry to obtain the mass pattern of the peptide products (Tab. 1 and FIG. 2) and by nanoflow LC-ESI mass spectrometry in the MS/MS mode to get sequence information (FIG. 3).

Native MUC1 samples and the recombinant fusion protein did not yield detectable amounts of peptide fragments (Tab. 1). On the other hand, the non-glycosylated pentameric repeat peptide (100mer) and the glycopeptides based on the AHG21 sequence were extensively fragmented (Tab. 1, FIG. 2). The 100mer yielded two major fragments with relative masses at m/z 1888.0 and 1630.0 corresponding to the GVT20 and SAP17 peptides derived from the MUC1 repeat sequence (Tab. 1). The AHG21 glycopeptides AHGVTSAPD(E)T(S)RPAPGSTAPP(A)A (substituted with one GalNAc residue) were identified at m/z 2249.0 and 2223.0, respectively, corresponding to the masses of N-thiopropionylated H1 to H3 (m/z 2249.0) and H4 to H6 (m/z 2223.0). The only products identified were registered at m/z 1695.7 (P1) and m/z 1669.7 (P2), respectively, corresponding to the GalNAc containing peptide fragments SAP16. The sequence of the two peptide products (P1, P2) were confirmed by MS/MS on a Qtof2 instrument to comprize 16 aa long C-terminal portions of the AHG21 glycopeptides (FIG. 3),

| | | |
|---|---|---|
| P1 | SAPDTRPAPGSTAPPA | SEQ ID NO: 7 |
| and | | |
| P2 | SAPESRPAPGSTAPAA, | SEQ ID NO: 8 |

both containing GalNAc at Thr/Ser10 or Thr17 (numbering according to the AHG21 sequence). No SAP16 peptides devoid of GalNAc were registered at m/z 1492 and 1466, respectively, indicating that proteolysis of AHG21 with GalNAc at Thr5 adjacent to the cleavage site had not occurred and that GalNAc had not been removed prior to proteolysis. The five aa N-terminal proteolytic fragment AHGVT (N-thiopropionylated) was not detected in any of the spectra Control experiments with DC primed AIMV media without antigen indicated that no proteolytic activity had been secreted into the medium, since no endopeptidase cleavage of TAP25 peptide was detected after 24 h incubation at 37° C. However, after adjustment of the supernatant to conditions optimal for cystein proteases (pH 5.5, 1 mM dithiothreitol) minor exopeptidase cleavage of the peptide was registered in the mass spectrum. Hence, the SAP16 fragments detected in the supernatants of antigen-pulsed cells can be regarded as cellular products and not as extracellular products of secreted proteases.

TABLE 1

Cellular processing products of native MUC1 and
MUC1 glycopeptides in mouse dendritic cells

| | | |
|---|---|---|
| | | Average mass |
| | | of peptide |
| | | fragments |
| | Structure | (structural |
| Antigen^{a} | (repeat number) | assignment) |

| | | | |
|---|---|---|---|
| 100mer | (HGVTSAPDTRPAPGSTAPPA)₅ (5) | 1629.8 | (SAP17) |
| Asc-MUC1 | (polymeric) | — | |
| MFP6 | (6) | — | |
| | [symbol] | | |
| H1 | AHGVTSAPDTRPAPGSTAPPA (1) | — | |
| | [symbol] | | |
| H2 | AHGVTSAPDTRPAPGSTAPPA (1) | 1695.6 | (SAP16 + |
| | | | HexNAc) |
| | [symbol] | | |
| H3 | AHGVTSAPDTRPAPGSTAPPA (1) | 1695.6 | (SAP16 + |
| | | | HexNAc) |
| | [symbol] | | |
| H4 | AHGVTSAPESRPAPGSTAPAA (1) | | — |
| | [symbol] | | |
| H5 | AHGVTSAPESRPAPGSTAPAA (1) | 1669.6 | (SAP16 + |
| | | | HexNAc) |
| | [symbol] | | |
| H6 | AHGVTSAPESRPAPGSTAPAA (1) | 1669.6 | (SAP16 + |
| | | | HexNAc) |

^{a}Asc-MUC1, MUC1 from pooled human tumor ascites; MFP6; MUC1 fusion protein expressed in human embryonic kidney cell line EBNA 293 (13); biotinylated glycopeptides H1 to H6 with defined glycosylation sites: [symbol] GalNAc

### Example 3

### In Vitro Proteolysis of Native MUC1 and MUC1 Glycopeptides with Human Cathepsin L Coincides with Cellular Processing

In Vitro Proteolysis of Native MUC1 and MUC1 (Glyco)Peptides with Human Cathepsins

Human cathepsins L and D were purchased from Sigma (München, Germany) and solubilized in 0.1 M sodium acetate buffer, pH 5.5, containing 1 mM EDTA (cathepsin D) and 1 mM dithiotreitol (cathepsin L). 2-5 units of enzyme(s) were added to 100 μg of mucin or recombinant fusion protein or to 10 μg of (glyco)peptide substrates in a total volume of 20 μl digestion buffer (see above). The reaction mixtures were incubated at 37° C. and 2 μl were withdrawn after 3 h or 24 h and diluted 20 fold in 0.1% aqueous TFA prior to MALDI mass spectrometry. In case of native MUC1 samples with complex O-glycosylation (MUC1 from tumor ascites, HMFG-MUC1, partially deglycosylated HMFG-MUC1, and MFP6 fusion protein) the digest was desalted by solid-phase extraction (ZipTip C18) and the (glyco)peptides were deglycosylated by β-elimination/ethylaminylation in 70% ethylamine at 50° C. as previously described (Müller and Hanisch, J. Biol. Chem. 277 (2002), 26103-26112). Each glycosylated position in the peptide fragments corresponds to the addition of a 27 u mass increment.

3.1 Monomeric Repeat Peptides and Glycopeptides

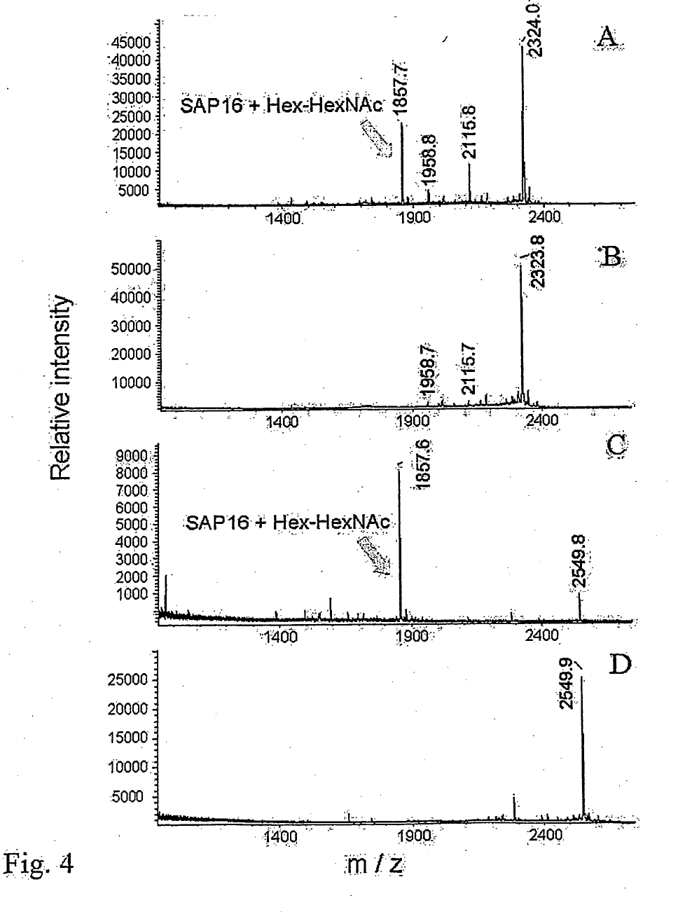

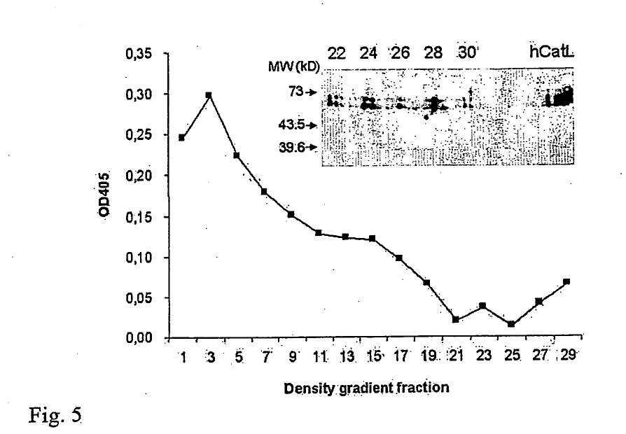

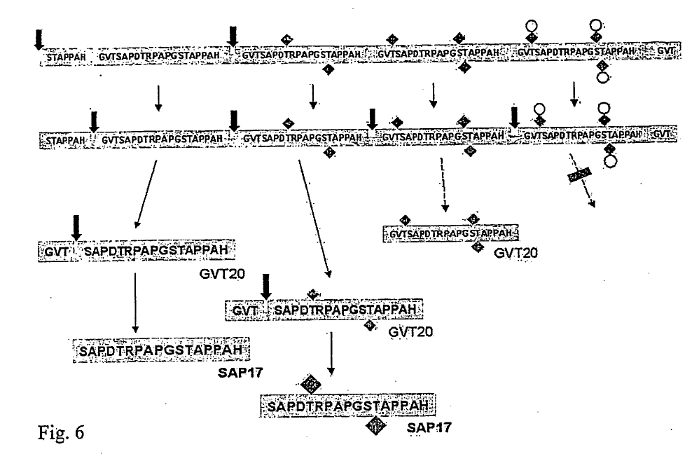

To verify the processing data obtained with human and mouse DCs and to confirm the proposed identity of the preferentially involved protease(s) we performed a series of in vitro digestions with cathepsin L and selected MUC1 protein and (glyco)peptide substrates (Tab. 2, and 3, FIG. 4). Using standard conditions for cysteine proteases and incubation times of 3 h the enzyme was able to cleave all non-glycosylated monomeric MUC1 repeat peptides quantitatively, except for the variant GST20-AES peptide (80% cleavage). Irrespective of the N-terminal tripeptide motif in the repeat sequence (TAP, AHG, GST) and of the length of the peptides (20mer, 21mer, 25mer), human cathepsin L cleaved specifically between Thr-Ser in the VTSA motif of the repeat peptide (Tab. 2). Besides this preferential cleavage site, which is in accordance with the cellular processing, minor activities of the enzyme preparation were found to be directed to the adjacent positions Val-Thr (TSA17 at m/z 1958.8) and Ser-Ala (APD15 at m/z 1771.0). To examine the possibility that aminopeptidases could be responsible for the generation of these minor products, a protected substrate carrying a biotin label at the amino terminus was used as substrate (FIG. 4). The terminally protected glycopeptide showed only one major product at m/z 1858.7 corresponding to the glycosylated SAP16 fragment (FIG. 4C). Catalytic activity of the cystein endopeptidase directed to the Thr-Ser bond was specifically inhibited with 1 μM Leu-Leu-Leu fluoromethyl ketone, while minor aminopeptidase activity in the cathepsin L preparation remained unaffected (FIG. 4B). In agreement with DC-mediated processing was the finding that O-glycosylated peptides, carrying GalNAc or Galβ1-3GalNAc, were effectively digested (Tab. 2, FIGS. 4A,C). However, the position of glycan attachment to one of the tree threonines (Thr5, Thr10, Thr17) in the AHG21 sequence was found to be critical as suggested by results from cellular processing. While GalNAc or Galβ1-3GalNAc in positions more distant from the cleavage site (Thr10, Thr17) had no influence on the cleavage by cathepsin L, the glycopeptides H1 and H4, both being glycosylated at the cleavage site (Thr5), were stable to proteolysis. Minor exopeptidase activity was detectable (in case of these glycopeptides) in the cathepsin L preparation from human liver.

TABLE 2
In vitro proteolysis of MUC1 glycopeptides and peptides with human cathepsin L

| | | |
|---|---|---|
| Substrate | Structure/Sequence | Average mass of production (mass range 500-4500 Da) |

Synthetic monomeric repeat peptides

| | | |
|---|---|---|
| H1 | 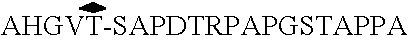 | 2162.8 (AHG21 + HexNAc) |
| H2 | 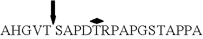 | 1696.5 (SAP16 + HexNAc) |
| H3 | 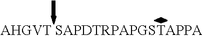 | 1696.7 (SAP16 + HexNAc) |
| A3 | 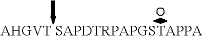 | 1858.0 (SAP16 + Hex-HexNAc) |
| TAP25 SEQ ID NO: 9 | 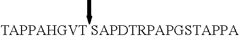 | 1491.8 (SAP16) |
| GST20-AES SEQ ID NO: 10 | 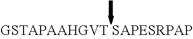 | 910.8 (SAP9) |

TABLE 3
In vitro proteolysis of MUC1 glycopeptides and peptides with human cathepsin L

| Substrate | Structure/Sequence (repeat number) | Average mass of repeat fragment ion (structural assignment) |
|---|---|---|
| Native mucin | | |
| HMFG-MUC1^{a} | (polymeric) | —^{b} |
| GalNAc-MUC1^{a} | (polymeric) | 1915.7, 1942.7, 1969.7, 1996.8 (GVT20 + 1-4 HexNAc)^{b} |
| Asc-MUC1^{a} | (polymeric) | —^{b} |
| MFP6^{a} | (6) | —^{b} |

Synthetic oligomeric peptides

| | | |
|---|---|---|
| 100mer | 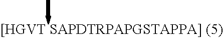 | 1888.0 (GVT20), 1629.5 (SAP17) |
| Tn-100mer | 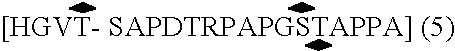 | 2498.1 (GVT20 + 3 HexNAc) |
| Tf-100mer | 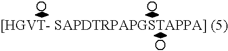 | — |

3.2 Mucins and Oligomeric Repeat Peptides and Glycopeptides

Oligomeric MUC1 repeat domains with complex and dense O-glycosylation (native mucin from HMFGs and tumor ascites, and recombinant fusion protein) were not digested by cathepsin L (Tab. 3). On the other hand, after partial de-O-glycosylation of HMFG-MUC1 by controlled chemical cleavage of peripheral and backbone sugars (Müller et al., J. Biol. Chem. 272 (1997), 24780-24793) the derivative with residual GalNAc substitution revealed fragmentation by cathepsin L at low efficiency (Tab. 3). The products (registered at m/z 1915.7, 1942.7, 1969.7, and 1996.8), which were detectable after β-elimination of GalNAc and Michael addition of ethylamine (Hanisch et al., Anal. Biochem. 290: 47-59, 2001), correspond to 20meric peptides of the MUC1 repeat domain carrying one to four substituents. Sequencing by ESI-MS/MS revealed that the 20meric peptide started with the GVT motif. The same peptide product was detected on digestion of GalNAc-substituted 100mer (Tn-100mer) carrying three sugar residues per repeat at each of the threonines. However, the cathepsin L catalyzed fragmentation of Tn-100mer resulted in the formation of only tiny amounts of glycosylated GVT20 (Tab. 3). Using the derived Tf-100mer with two to three corel disaccharides per repeat no proteolytic fragmentation was registered (Tab. 3).

Contrasting to the glycopeptides, the non-glycosylated 100mer peptide was extensively degraded in vitro resulting in the intermediate formation of GVT20 peptides (dominating after 3 h), which were finally cleaved (during 24 h) at the Thr-Ser bond to yield SAP17 (Tab. 3). These findings point to a general inhibition of proteolytic activity by O-glycosylation being apparent already on the monosaccharide level, but even more strongly on the disaccharide or higher levels of complexity.

As a control, human cathepsin D was tested with a selected panel of MUC1 repeat peptides and glycopeptides and found to be unable to use any of these as a substrate, even if incubation times of up to 24 h were chosen (Tab. 4). It can be concluded that proteolytic activity in the human cathepsin L preparation recapitulated all major aspects of MUC1 glycopeptide processing in human and mouse DCs.

TABLE 4

In vitro proteolysis of MUC1 glycopeptides and
peptides with human cathepsin D

| | | Average mass of |
|---|---|---|
| | | production |
| Sub- | Structure/Sequence | (mass range |
| strate | (repeat number) | 500-4500 Da) |
| | [symbol] | |
| H1 | AHGVT-SAPDTRPAPGSTAPPA | 2161.9 |
| | | (AHG21 + HexNAc) |
| | [symbol] | |
| H2 | AHGVT-SAPDTRPAPGSTAPPA | 2161.9 |
| | | (AHG21 + HexNAc) |
| | [symbol] | |
| H3 | AHGVT-SAPDTRPAPGSTAPPA | 2161.9 |
| | | (AHG21 + HexNAc) |
| 100mer | [HGVT-SAPDTRPAPGSTAPPA] (5) | — |

The substrates (10 to 100 μg in 20 μl 0.1 M sodium acetate buffer, pH 5,5. containing 1 mM EDTA were incubated with 0 cathepsin D for 24 h or with cathepsin L for 3 h (in the presence of 1 mM DTT) at 37° C. [symbol] refers to O-linked GalNAc, [symbol] to O-linked Galβ1-3GalNAc.
^{a}HMFG-MUC1, mucin from human milk, fat globule membranes, GalNAc-MUC1. partially deglycosylated HMFG-MUC1; Asc-MUC1, mucin from pooled tumor ascites; MFP6, recombinant fusion protein expressed in the human embryonic kidney cell line EBNA-293; H1, H2, H3, A3, TAP25 and GST20-AES represent N-terminally unmodified (glyco)peptides.
^{b}Mass spectra were recorded after de-O-glycosylation/ethylaminylation (12).

### Example 4

### In Vitro Proteolysis of MUC1 Glycopeptides with Enzymes in Low-Density Endosomal Fractions from Mouse Dendritic Cells Coincides with Cellular Processing

Mouse dendritic cells (10⁸) were homogenized by fine-needle aspiration on ice using 1 ml of 0.3 M sucrose, 0.01 M Hepes as buffer (without protease inhibitors). After dilution to 7 ml and centrifugation at 850 g for 10 min to remove intact cells and nuclei, 6 ml of the supernatant were centrifuged over 24 ml of 30% Percoll with 0.3 M sucrose, 0.01 M Hepes for 105 min at 20.000 rpm in a centrifuge (model J2-21 M/E, rotor: JA-20, Beckman instruments, München, Germany) (Barnes et al., J. Exp. Med. 181 (1995), 1715-1727). The gradient was fractionated by gravity siphon (30×1 ml) and each fraction was analysed after sonication for the presence of MHC class II molecules by enzyme immunoassay with anti-H2 antibody (rat hybridoma cell line M1/42.3.9.8.HLK obtained from the ATCC), β-hexosamimidase activity (Barnes et al., 1995) and cathepsin L related proteolytic activity using TAP25 peptide as substrate (5 μg). The samples were incubated for 24 h at 37° C., diluted 20 fold in aqueous TFA and analysed by MALDI mass spectrometry. For specific inhibition of cathepsin L activity, the corresponding fractions were mixed with 1 μM Z-Leu-Leu-Leu-fluoromethyl ketone (Sigma). Westernblot analyses of 290 μl aliquots of the density gradient fractions were performed under standard conditions (Müller et al., J. Biol. Chem. 274 (1999), 18165-18172) using anti mouse cathepsin L antibody CPLH 3G10 (Alexis Biochemicals, Grünberg, Germany).

Mouse dendritic cells were ruptured in the absence of protease inhibitors and the supernatant (after removal of nuclei) was centrifuged in a Percoll gradient. The gradient fractions were tested for proteolytic activity using TAP25 as a substrate and incubation conditions were optimized for cysteine proteases (FIG. 5). Low density endosomes were separated from lysosomes according to the registration of marker proteins (β-hexosamimidase) (FIG. 5) and demonstrated to contain a cysteine protease inhibitable with Z-Leu-Leu-Leu-fluoromethyl ketone and with a site-specificity related to human cathepsin L.

Mouse cathepsin L was identified in low density fractions (fractions 22 to 30) by westernblot analysis using a monoclonal antibody (FIG. 5, insert). Cathepsin L-like enzymatic activity was isographic with these positively stained fractions, since enzymes in fractions with a density of approx. 1.037 g/ml cleaved TAP25 peptide at Thr-Ser yielding SAP16, while all other fractions, in particular those with densities above 1.054 g/ml, contained no such activity, but considerable activities of carboxypeptidase(s).

The presented work for the first time reveals insight into the molecular aspects of processing by human and murine DCs of the human glycoprotein tumor antigen MUC1, a mucin, which could serve as a model for processing of other heavily O-glycosylated antigens. Using state-of-the-art methodologies for the structural characterization of (glyco)peptides this study was able to answer four important questions regarding MUC1 proteolysis by APCs in the MHC class II pathway: 1) Where are the cleavage sites in the MUC1 repeat peptide? 2) In which way do O-linked glycans affect proteolytic cleavage? 3) Are core-type glycans removed prior to proteolytic processing? 4) Which of the enzymes involved in the processing machinery are responsible for proteolytic cleavage of MUC1 repeat peptides? The results obtained in accordance with the present invention suggest that MUC1 repeats are cleaved mainly at two sites, at the His-Gly bond and between Thr-Ser in the VTSA motif (FIG. 6). During cellular processing the core-type glycans GalNAc and Galβ1-3-GalNAc were not removed (see also Vlad et al., J. Exp. Med. 196 (2002), 1435-1446,), but inhibited the cleavage if they were located adjacent to the cleavage site. The revealed molecular aspects, in particular the site specificity of cleavage and the site-dependent effects of carbohydrates coincided in the human and mouse system, and were in perfect agreement in the cellular and in vitro assays. Moreover, the cellular processing after pulsing of DCs with biotinylated bead-conjugated glycopeptides was in coincidence with the processing of native, untagged antigen in a soluble form. This series of coincidences reduces the likeliness of artefacts, which might have been introduced by the use of tagged immobilized antigen in some of the experiments.

The structural features of MUC1 processing products (FIG. 6) match findings from an immunological study (Vlad et al., J. Exp. Med. 196 (2002), 1435-1446). According to this work, glycans remain intact during processing of MUC1 glycopeptides by DCs, but influence activation of T cell hybridoma clones in a site-specific manner. Clone VF5, reactive to a peptide epitope that comprizes the DTR motif, was activated by DCs pulsed with AHG21 glycopeptides which carried glycans at Ser16 or Thr17. No activation of this clone was measurable, however, if the glycans were located at the proposed epitope or at the Thr/Ser positions adjacent to the cathepsin L cleavage site defined in the present study (Thr5-Ser6). Hence the O-linked glycans can alter proteolytic processing or presentation of the MHC class II-restricted glycopeptides in a site-specific manner. While glycans alter processing of glycopeptides they do not always affect binding of processed glycopeptides by MHC class II, as was demonstrated previously (Jensen et al, J. Immunol. 158 (1997), 3769-3778).

Cathepsin L, a cysteine protease related to papain, has been claimed to be involved in antigen processing (Nakagawa et al., Immunol. Rev. 172 (1999), 121-129; Honey et al., J. Biol. Chem. 276 (2001), 22573-22578). We confirmed the possible involvement of cathepsin L (or a closely related enzyme species) in MUC1 repeat proteolysis by specific inhibition of the human and mouse enzyme. The in vitro data with cathepsin L show that oligomeric tandem repeats are fragmented by the enzyme to intermediate GVT20 peptides (FIG. 6), a process which is not site-controlled, but quantitatively affected by O-glycosylation. Accordingly, substitution with three GalNAc residues reduces proteolytic activity, and substitution with three Gal-GalNAc disaccharides results in no detectable fragmentation (FIG. 6). The initially formed GVT20 peptides are finally degraded to SAP17 under the prerequisite that Thr in the VTSA motif is not substituted with a glycan. This site is, however, a preferred target for the ubiquitously expressed polypeptide GalNAc-transferases, ppGalNAc-T1 and -T2, (which were used for in vitro glycosylation of 100mer peptide). In agreement with this, the Tn-100mer and the densely O-glycosylated native mucin samples were characterized in previous studies (Müller et al., S. Biol. Chem. 272 (1997), 24780-24793; Beatty et al., Clin. Cancer Res. 7 (2001), 781-787) to carry glycans at this site throughout (Tn-100mer) or in the majority of the tandem repeats (native MUC1 from cancer cells). Non-glycosylation of the cathepsin L cleavage site in two adjacent repeats should, accordingly, be rare and the formation of SAP17 fragments from these mucin samples unlikely. The highly specific processing of MUC1 and the concomittant restriction of effective proteolytic cleavage to particular glycoforms of the mucin repeat domain would explain the weak immunogenicity of native MUC1 from milk fat membranes or from tumor ascites as related to the weak T cell responses observed in previous study (Vlad et al., J. Exp. Med. 196 (2002), 1435-1446). The masking of potential processing sites by O-glycosylation might also be a new mechanism on the level of posttranslational protein modification to avoid autoimmunity against otherwise immunogenic protein backbones.

Although cathepsin L may not be required for the generation of a majority of epitopes it can strongly affect the generation of a subset of antigenic epitopes in both a positive and a negative fashion suggesting a direct role for this protease, but also for the related cathepsin S, in antigen processing (Hsieh et al., J. Immunol. 168 (2002), 2618-2625). It can be anticipated that antigen processing in late endosomes is mediated by a family of proteases with partially overlapping, but still distinct specificities. Hence, the in vitro data on cathepsin L cleavage of MUC1 presented in this paper do not exclude the possible involvement of other, cathepsin L related, enzymes in the processing machinery and in the specific cleavage of MUC1. Sequence data suggest that cathepsins L and S are the most closely related enzymes in this family (Santamaria et al., J. Biol. Chem. 274 (1999), 13800-13809), if a recently identified isoform of cathepsin L, cathepsin L2, is not taken into account (Santamaria et al., Cancer Res. 58 (1998), 1624-1630). Sequence homology and identical cleavage patterns observed in cellular processing by human or mouse DCs and in vitro with human cathepsin L support the assumption that the enzymes in both species exhibit similar specificities.

Tumor-associated MUC1, in particular the glycoforms from breast cancer cells, have been claimed to exhibit underglycosylated protein cores (Lloyd et al., J. Biol. Chem. 271 (1996), 33325-33334), referring to both, to truncated chain lengths and to a reduced number of glycosylated sites per repeat. Recently, it could be shown that this finding cannot be transferred to secreted mucin, since the structural analysis of MUC1 samples that were recombinantly expressed in four different breast cancer cell lines revealed increased substitution densities with complex, individually fluctuating O-glycans (Müller et al., J. Biol. Chem. 277 (2002), 26103-26112). An average profile of O-linked glycans determined for MUC1 from pooled ascites samples of breast and pancreatic cancer patients was also in accord with a more complex glycosylation (Jensen et al., J. Immunol. 158 (1997), 3769-3778). In agreement with our findings, this glycoform of the mucin represents a weak immunogen in the MHC class I pathway (Hiltbold et al., Cell. Immunol. 194 (1999), 143-149) and is non-immunogenic in the MHC class II pathway (Hiltbold et al., J. Immunol. 165 (2000), 3730-3741). The latter phenomenon has been assigned to an entrapment in early endosomes of DCs mediated by multivalent, high-avidity interaction with the mannose receptor (Hiltbold et al., J. Immunol. 165 (2000), 3730-3741). It can be concluded, accordingly, that O-glycosylation of MUC1 interferes first of all with trafficking of endocytosed mucin. Later on, if late endosomal compartments are accessible for the antigen, other modes of interference mediated by O-linked glycans could also come into play, like inhibition of proteolysis. The existence of such restrictions introduced by site-specific O-glycosylation became evident in the present study, since glycans linked to Thr/Ser in the VTSA motif of MUC1 repeats prevented processing of the glycopeptides (FIG. 6). The site-specificity of glycan substitution has to be considered in the design of synthetic cancer vaccines. To make glycosylated epitopes available for MHC class II presentation, it might be advantageous to use synthetic glycopeptides lacking glycosylation at the VTSA motif. Moreover, SAP17 peptides and their glycosylated derivatives may represent a “pre-processed” form suitable for external loading on MHC class II molecules in immunotherapeutic approaches. In loading experiments with SAP17 the glycosylation-dependent effects on the binding to MHC class II proteins and on recognition by the T cell receptors can now be studied by systematic variation of the substitution sites and structures of the glycans.

Hence, the present invention provides a novel approach for the design of immunogenic MUC1 peptides that can be used as anti-cancer vaccines.

1. A peptide of least 9 amino acids in length derived from the tandem repeat domain of MUC1 and having the amino acid sequence SAP at its N-terminus.

2. The peptide of claim 1, wherein said peptide comprises of 10 to 25 amino acids of said tandem repeat domain of MUC1.

3. The peptide of claim 1 or 2, which is a fragment of said tandem repeat domain.

4. The peptide of claim 1, which comprises an amino acid sequence of any one of SEQ ID NOS: 1 to 4 or 11, or variants thereof, wherein said variants comprise one or more amino acid additions, insertions, substitutions or deletions as compared to the sequence of any one of SEQ ID NOS: 1 to 4 or 11, and wherein the biological activity of said peptide is substantially equal to the activity of the peptide comprising the unmodified amino acid sequence of any one of SEQ ID NOS: 1 to 4 or 11.

5. The peptide of claim 1, wherein one or more threonines or serines of the peptide are O-glycosylated.

6. The peptide of claim 5, having an amino acid sequence of any one of SEQ ID NOS: 1 to 4 or 11, wherein the amino acid is glycosylated at Thr 5 or 12.

7-24. (canceled)

25. The peptide of claim 3, wherein said peptide is SAP17 (SEQ ID NO: 11).

26. A nucleic acid encoding a peptide of claim 1.

27. A method of producing a peptide having at least 9 amino acids derived from the tandem repeat domain of MUC1 and having the amino acid sequence SAP at its N-terminus, comprising the following steps:
(a) providing a peptide comprising the tandem repeat domain of MUC1 or a part thereof, which part at least contains one repeating unit of said tandem repeat domain of MUC1;
(b) contacting the peptide of (a) with an effective amount of cathepsin-L or a closely related enzyme hereof, thereby cleaving the peptide; and
(c) isolating the fragments produced in (b).

28. The method of claim 27, wherein the peptide provided in step (a) is natural MUC1 derived from human milk fat membranes, from human tumor ascites, or from human breast carcinoma cell lines or is represented by any one of SEQ ID NOS: 5, 6, 9, 10, or 12.

29. The method of claim 27, wherein one or more of the amino acids of the peptide provided in step (a) is O-glycosylated, provided that the peptide is not glycosylated at the cleaving site of cathepsin-L.

30. The method of claim 27, wherein one or more threonines or serines of the peptide fragment isolated in (c) are O-glycosylated.

31. A peptide obtainable by the method of claim 27.

32. A fusion molecule comprising the peptide of claim 31.

33. An ex vivo-method of producing a population of autologous antigen presenting cells (APCs), which are capable of inducing effective immune responses against MUC1, comprising the steps of
(a) providing autologous APCs from a tumor patient;
(b) contacting the autologous APCs from the tumor patient with an effective amount of a peptide or fusion molecule comprising at least 9 amino acids of the tandem repeat domain of MUC1 and having the amino acid sequence SAP at its N-terminus, wherein said contacting is under conditions which allow endocytosis, processing, and MHC class II presentation of fragments of said peptide or fusion molecule by said APCs; and
(c) isolating said peptide or fusion molecule fragment-presenting APCs for the purpose of immunotherapeutic application in the patient.

34. The method of claim 33, wherein said peptide or fusion molecule comprises 10 to 25 amino acids of said tandem repeat domain of MUC1.

35. The method of claim 33, wherein said peptide or fusion molecule is a fragment of said tandem repeat domain.

36. The method of claim 35, wherein said peptide or fusion molecule is SAP17 (SEQ ID NO: 11).

37. The method of claim 33, wherein said peptide or fusion molecule comprises an amino acid sequence of any one of SEQ ID NOS: 1 to 4 or 11, or variants thereof, wherein said variants comprise one or more amino acid additions, insertions, substitutions or deletions as compared to the sequence of any one of SEQ ID NOS: 1 to 4 or 11, and wherein the biological activity of said peptide or fusion molecule is substantially equal to the activity of the peptide or fusion molecule comprising the unmodified amino acid sequence of any one of SEQ ID NOS: 1 to 4 or 11.

38. The method of 33, wherein one or more threonines or serines of said peptide or fusion molecule are O-glycosylated.

39. The method of claim 38, wherein said peptide or fusion molecule has an amino acid sequence of any one of SEQ ID NOS: 1 to 4 or 11, wherein the amino acid is glycosylated at Thr 5 or 12.

40. The method of claim 33, wherein the peptides or fusion molecules in (b) are bound to ferric oxide beads.

41. An ex vivo-method of producing genetically engineered antigen presenting cells (APCs), which are capable of inducing effective immune responses against MUC1, comprising the steps of
(a) providing a nucleic acid encoding a peptide or fusion molecule comprising at least 9 amino acids of the tandem repeat domain of MUC1 and having the amino acid sequence SAP at its N-terminus,
(b) transfecting the APCs with said nucleic acid, and
(c) selecting APCs, which present said peptides in an MHC II restricted manner.

42. The method of claim 41, wherein the nucleic acid of step (a) is provided in an expression vector.

43. The method of claim 41, wherein said peptide comprises 10 to 25 amino acids of said tandem repeat domain of MUC1.

44. The method of claim 41, wherein said peptide is a fragment of said tandem repeat domain.

45. The method of claim 41, wherein said peptide is SAP17 (SEQ ID NO: 11).

46. The method of claim 41, wherein said peptide comprises an amino acid sequence of any one of SEQ ID NOS: 1 to 4 or 11, or variants thereof, wherein said variants comprise one or more amino acid additions, insertions, substitutions or deletions as compared to the sequence of any one of SEQ ID NOS: 1 to 4 or 11, and wherein the biological activity of said peptide is substantially equal to the activity of the peptide comprising the unmodified amino acid sequence of any one of SEQ ID NOS: 1 to 4 or 11.

47. The method of 41, wherein one or more threonines or serines of said peptide are O-glycosylated.

48. The method of claim 47, wherein said peptide has an amino acid sequence of any one of SEQ ID NOS: 1 to 4 or 11, wherein the amino acid is glycosylated at Thr 5 or 12.

49. An antigen presenting cell (APC) obtainable by the method of claim 41.

50. The APC of claim 49, which is a dendritic cell or a B cell.

51. A composition comprising a therapeutically effective amount of a peptide or fusion molecule comprising at least 9 amino acids of the tandem repeat domain of MUC1 add having the amino acid sequence SAP at its N-terminus or an antigen presenting cell comprising said peptide or fusion molecule and a pharmaceutically acceptable carrier.

52. The composition of claim 51, wherein said peptide or fusion molecule comprises 10 to 25 amino acids of the tandem repeat domain of MUC1.

53. The composition of claim 51, wherein said peptide or fusion molecule is a fragment of said tandem repeat domain.

54. The composition of claim 53, wherein said peptide or fusion molecule is SAP17 (SEQ ID NO: 11).

55. The composition of claim 51, wherein said peptide or fusion molecule comprises an amino acid sequence of any one of SEQ ID NOS: 1 to 4 or 11, or variants thereof, wherein said variants comprise one or more amino acid additions, insertions, substitutions or deletions as compared to the sequence of any one of SEQ ID NOS: 1 to 4 or 11, and wherein the biological activity of said peptide or fusion molecule is substantially equal to the activity of the peptide or fusion molecule comprising the unmodified amino acid sequence of any one of SEQ ID NOS: 1 to 4 or 11.

56. The composition of 51, wherein one or more threonines or serines of said peptide or fusion molecule are O-glycosylated.

57. The composition of claim 56, wherein said peptide or fusion molecule has an amino acid sequence of any one of SEQ ID NOS: 1 to 4 or 11, wherein the amino acid is glycosylated at Thr 5 or 12.

58. The composition of claim 51, which is a vaccine.

59. A method of treating a patient suffering from a MUC1-positive carcinoma, said method comprising administering a composition comprising a peptide or fusion molecule comprising at least 9 amino acids of the tandem repeat domain of MUC1 and having the amino acid sequence SAP at its N-terminus or an antigen presenting cell comprising said peptide or fusion molecule, wherein said composition is administered to said patient in an amount effective to induce an immune response against MUC1.

60. The method of claim 59, wherein said peptide or fusion molecule comprises 10 to 25 amino acids of the tandem repeat domain of MUC1.

61. The method of claim 59, wherein said peptide or fusion molecule is a fragment of said tandem repeat domain.

62. The method of claim 61, wherein said peptide or fusion molecule is SAP17 (SEQ ID NO: 11).

63. The method of claim 59, wherein said peptide or fusion molecule comprises an amino acid sequence of any one of SEQ ID NOS: 1 to 4 or 11, or variants thereof, wherein said variants comprise one or more amino acid additions, insertions, substitutions or deletions as compared to the sequence of any one of SEQ ID NOS: 1 to 4 or 11, and wherein the biological activity of said peptide or fusion molecule is substantially equal to the activity of the peptide or fusion molecule comprising the unmodified amino acid sequence of any one of SEQ ID NOS: 1 to 4 or 11.

64. The method of 59, wherein one or more threonines or serines of said peptide or fusion molecule are O-glycosylated.

65. The method of claim 64, wherein said peptide or fusion molecule has an amino acid sequence of any one of SEQ ID NOS: 1 to 4 or 11, wherein the amino acid is glycosylated at Thr 5 or 12.

66. The method of claim 59, wherein said composition is a vaccine.

67. The method of claim 59, wherein the MUC1-positive carcinoma is a breast, a colorectal, a pancreatic or a gastric cancer.
